(19) United States
(12) Patent Application Publication
(10) Pub. No.: US 2005/0165405 A1
(43) Pub. Date: Jul. 28, 2005

(54) Minimal access apparatus for endoscopic spinal surgery

(76) Inventor: Paul M. Tsou, Santa Monica, CA (US)

Correspondence Address: MARVIN H. KLEINBEG;KLEINBERG & LERNER, LLP, Suite 1080, 2049 Century Park East, Los Angeles, CA, 90067, US

(21) Appl. No.: 10/749,457

(22) Filed: Dec. 31, 2003

Related U.S. Application Data
(63) Continuation-in-part of application No. 09/997,361, filed on Nov. 30, 2001, now Pat. No. 6,851,430.

(51) Int. Cl. A61B 19/00

(52) U.S. Cl. 606/86

(57) ABSTRACT

A system for minimal access soft tissue dilating and retracting and nucleus pulposus excision tools for endoscopic spinal surgery, includes elements to seek the appropriate trajectory, creation of soft tissue tunnel space, and retractors for the tunnels.

### BACKGROUND OF THE INVENTION

1. Field of the Invention

The present invention relates generally to minimal access apparatus for percutaneous surgery and, more specifically, to apparatus for performing endoscopic posterolateral transforaminal lumbar and thoracic disc surgery and interbody fusion.

2. Description of Prior Art

A substantial segment of the population suffers from axial spinal and or leg pain that are caused by degenerative, herniated and protruded intervertebral discs. Intervertebral discs are members of the spinal column that serve as cushions and mobile linkage elements between the individual vertebrae. The acute herniation of an intervertebral disc can lead to the compression of spinal nerve elements within the spinal canal as well as nerves located just outside of the spinal canal. The abnormal states will likely to cause severe back pain, leg pain, muscle weakness, and possibly bowel and bladder dysfunction.

The traditional surgical method of spinal nerve element decompression is by the posterior transcanal or transforaminal open approach. Laminectomy and facetectomy are required to gain entry into the spinal canal and disc space. Two blades soft tissue retractor/spreader is commonly used to maintain exposure leading to the lamina of the target level. The size of the typical skin incision is two to four inches for a single level disc surgery. More recently smaller diameter tubular retractors, which have non-tapered ends, have become available. Prior art soft tissue retractors remain positioned superficial to the lamina. Additional A different type of retractor is needed when surgical maneuver enters the spinal canal. Traditionally, this procedure has required two to three days of hospitalization after completion of the surgery.

Chronic back pain due to disc failure, without dominant extremity symptoms may also cause chronic functional impairment. Prior art solutions have surgically fused adjacent vertebrae together by placing bridging bone, or other osteoinductive and osteoconductive material from one vertebra above to one vertebrae below the symptomatic discs. The native bone fusion surfaces may include the posterior vertebral elements, the vertebral end plates or a combination of the two. Sometimes, metal rods and screws have been used to stabilize the spinal fusion segments from the posterior approach.

The invasive nature of prior art techniques cause significant access tissue trauma, even when the skin incision is reduced in length. The principle of minimal access surgery is to create the smallest possible cross sectional area tissue tunnel to the target pathology, without compromising the stabilizing structural elements. This reduces the amount of trauma suffered by the patient. At the same time, the minimal access tunnel needs to have the appropriate cross-sectional size and shape so that it can accommodate the transit of surgical tools and implants. The novel apparatus that create and maintain the minimal access tunnels are the inventions of this application.

Using endoscopic posterolateral transforaminal techniques, a surgeon can operate through the smallest (roughly 7 millimeters) possible tissue tunnel with visualizing endoscope and miniaturized tools for simple herniated disc excision. For interbody fusion a 10-16 mm. assembled multilateral angular access tunnel is invented for the delivery of structural graft and other implants. Because the access surgical trauma and destabilization are reduced with the minimal access technique, endoscopic posterolateral transforaminal surgery requires a shorter rehabilitation time.

The access approach is posterolateral transforaminal, lateral to the spinal canal. In using this approach, the risks of traumatizing nerve element and dural from sharp instruments and retraction are greatly reduced. The working soft tissue channel for simple herniated disc extraction is approximately 7 mm in diameter and the diameter is somewhat larger for fusion surgery. Because of the ultra miniaturization of the instruments, the procedure can be performed using local anesthetic agents and conscious sedation. Unlike prior art, overnight hospital stays are not necessary.

In order to fuse adjacent vertebrae, osteoinductive graft material is placed in the evacuated disc space between the bony end plates of the target vertebrae. After insertion of the structural graft material and any additional non-structural osteogenic agents, ingrowth of new autologous bone gradually replaces the graft material to create a unified structure that includes the first and last vertebrae in the fusion segment. Prior art techniques have used structural angular bone blocks, metallic cages, carbon fiber cages, hydrooxyapetite blocks or bone chips that are inserted into the intervertebral disc spaces. Prior art laparoscopic anterior lumbar fusion technique uses cylindrical bone dowel and metallic cages. These cylindrical shaped devices do not have optimal surface contact with the flat surface of the host end plate bed. Seating of a cylindrical/round shaped fillers requires end-plate cutting. Surgical end-plate cutting structurally weakens the end-plate and introduces the probability of implant fillers settling into the softer vertebral cancellous body. The preferred modular discoid shaped fillers provide maximum surface contact and do not need end-plate cutting for seating and stability.

Prior art lateral spinal approach, square shaped graft delivery tubes are bulky. The dimensions of block graft delivery via a prior art square tube do not take full advantage of the maximum outer dimensions of the delivery tube. Additionally, these prior art systems have no satisfactory method for graft insertions into the L5-S1 disc space. Because prior art minimally invasive systems require generally round tube delivery conduit, the subsequent graft shape is necessarily round/cylindrical as well.

One specific prior art technique, using a rounded filler, is discussed in U.S. Pat. No. 6,217,509 (the '509 patent). The '509 patent describes an access tubular channel from the skin to the targeted work area (which is only used in the posterior transcanal spinal approaches). The working channel inside the tube allows for the use, as needed, of a viewing element, operating tools, tissue retractors, and suction channel. This method is considered more problematic when used in any other approach. According to the '509 patent, a fluid working environment is not feasible in posterior lumbar surgery. However, a fluid environment is utilized in the present invention. Continuous ingress-egress of fluid aids in endoscopic vision during the ablation of bone, nucleus, collagenous tissue or bleeder coagulation. The fluid medium is made possible through the usage of Holmium-YAG laser in the present invention, which eliminates the problems that encountered by the '509 method. Heat and tissue debris are carried away from the laser strike zone in the continuous ingress-egress fluid environment.

Additionally, the '509 patent does not identify the necessary posterolateral skin entry location for instruments insertion nor can it enter into the intervertebral disc space. The present invention describes a skin window localization method, identified the safe foraminal annular window and the trajectory for the instruments. In addition the deep end of the preferred working cannulae are directly anchored in the opening of the annular window.

Finally, the '509 method neither describes nor allows for the delivery of modular discoid shaped bone and other osteoinductive, structural implant material (i.e., components of the module are rectangular or have round edges that face the interior of annulus fibrosus).

Therefore, what has been needed is a preferred shaped and sized minimal access apparatus. The apparatus permits a full spectrum of minimal access spinal surgery from nerve decompression, excision of herniated disc to delivery of structural implants.

### BRIEF SUMMARY OF THE INVENTION

According to the present invention, various apparatus are described whereby a surgeon can perform percutaneous endoscopic spinal disc surgery and introduce modular discoid shaped components as filler material for intervertebral fusion. In the preferred embodiment, the apparatus include innovative tools to create a tissue tunnel, retract soft tissue, and allow the insertion of the above-mentioned implants into the spinal intervertebral spaces.

### BRIEF DESCRIPTION OF THE DRAWINGS

FIG. 1 illustrates a transforaminal endoscopic excision technique for a paramedian disc herniation;

FIG. 2 illustrates a tapered spiral-end obturator of the present invention.

FIG. 3 illustrates a beveled canulum of the present invention.

FIG. 4 illustrates a flat blade spreader

FIG. 5 illustrates a cover that is used with the flat blade spreader of the present invention.

FIG. 6 illustrates a nucleus debriding tool.

FIG. 7 illustrates an abrasive tool for removing nucleus pulposus.

FIG. 8 illustrates a hollow shaving tool that is used to evacuate the intervertebral disc space.

### DETAILED DESCRIPTION OF THE INVENTION

According to the present invention, there is disclosed apparatus for performing percutaneous spinal posterolateral transforaminal endoscopic surgery including disc excision and interbody fusion using multilateral angularly shaped modular components. In the following description, for the purposes of explanation, specific devices, component arrangements and construction details are set forth in order to provide a more thorough understanding of the invention. It will be apparent to those skilled in the art, however, that the present invention may be practiced without these specifically enumerated details and that the preferred embodiment can be modified so as to provide other capabilities, such as the capability for the remote control to operate with other devices. In some instances, well-known structures and methods have not been described in detail so as not to obscure the present invention unnecessarily.

The present invention relates to minimal access apparatus and tools used during endoscopic spinal surgery. These tools are designed so as to enable a surgeon to more effectively perform the surgical techniques described herein and minimize trauma to a patient. In order to better understand the structure and operation of the tools, the preferred surgical methods will be briefly described. The preferred methodology is described in more detail in U.S. patent application Ser. No. 09/997,361. Although the tools of the present invention are specifically designed for use with the preferred surgical methods, they are not limited to those specific methods. Rather, the tools can be used in a variety of different surgical techniques and can be effectively used in a variety of operations which are not specifically described herein.

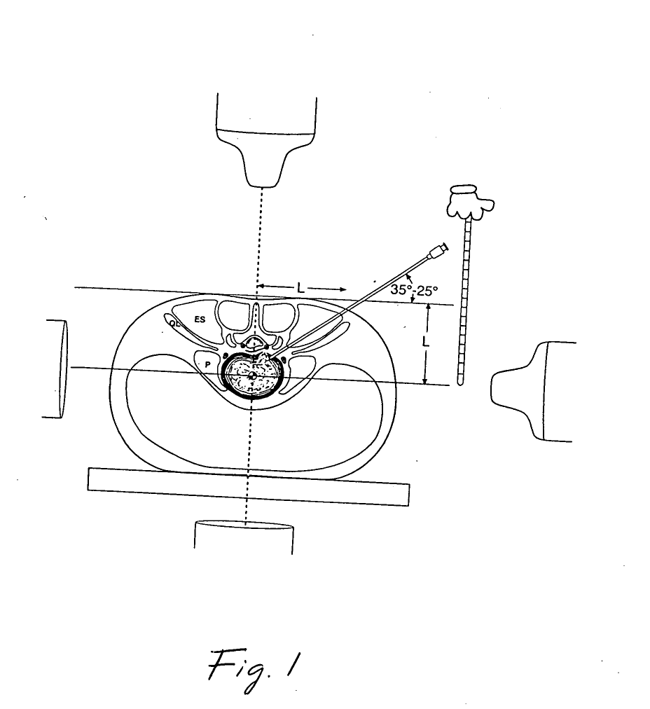

Referring First to FIG. 1, the preferred surgical methodology permits the patient to be awake during the procedure. A local anesthetic agent and conscious sedation are the method of analgesia. The skin window, subcutaneous tissue, muscle layer and trajectory tract are pierced with an approximately 6 inch long, 18-gauge needle and continuing towards the foraminal annular window. The skin window localization is determined by the index disc inclination and the measured length from the center of the disc to the posterior skin surface. The needle insertion trajectory is approximately 25-35 degrees in relationship to the body frontal plane in line with the disc inclination. After the foraminal annular window placement of the needle, a thin guide wire is inserted through the needle channel and advanced into the center of the disc.

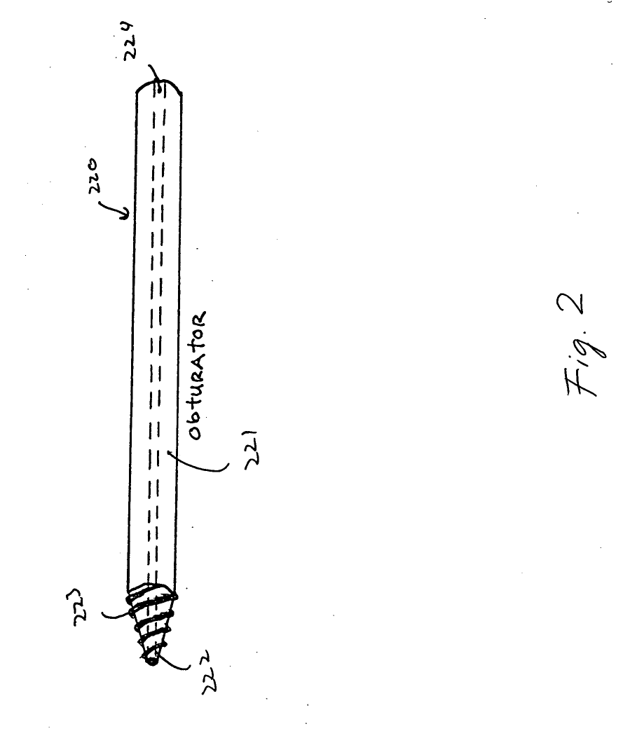

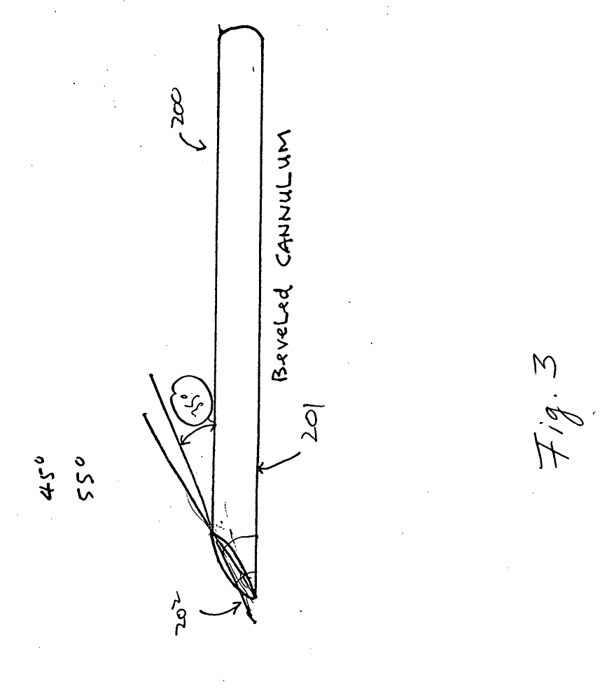

After the guide wire is accurately positioned, the needle is removed, a preferred tapered spiral-end obturator is introduced over the proximate tip of the guide wire and inserted toward the annulus at the foramen. (The tapered spiral-end obturator is described below with reference to FIG. 2.) The tapered spiral-end obturator is then advanced through the annulus at the foraminal location. The spiral tip of the obturator should be positioned within the annulus. The guide wire is then removed and the preferred beveled cannulum (described below with reference to FIG. 3), which has a larger oval viewing opening, is inserted over the obturator. Once the beveled tip of the cannulum is well within the annulus, the obturator is removed.

If the methodology is utilized to extract herniated spinal disc, herniated nucleus pulposus fragments are excised. In this instance, an operative endoscope is inserted. A working tunnel and cavity are created under the herniated elements to facilitate disc material removal.

When the spinal pathology requires a fusion procedure, the circular-shaped annular fenestration is enlarged by inserting a preferred tapered obturator/dilator of predetermined diameter. Once the obturator tapered end is deep inside the disc annulus, the surgeon inserts the preferred oval spreader over the obturator.

With the oval spreader deep end inside the disc, the annular opening is then further dilated. Progressively larger diameter solid rods are placed in the channel portion of the oval spreader until the opening is dilated to the largest anatomically feasible size.

Once the oval spreader has achieved maximum opening, excavation of the nucleus pulposus can be performed. Typically, this includes complete removal of the nucleus pulposus and the vertebral cartilaginous end plates to create a natural discoid shaped cavity for the placement of the preferred modular discoid shaped graft components. Nucleus pulposus can be removed using a variety of different tools, as will be described below.

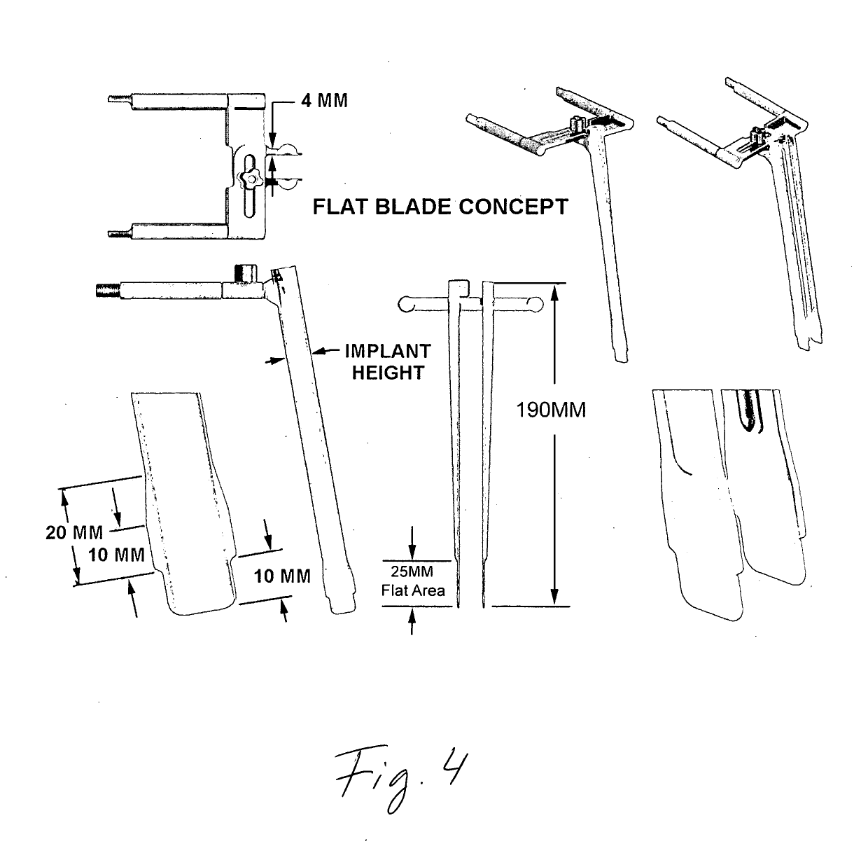

After the nucleus pulposus has been excavated from the intervertebral disc space, the preferred flat blade spreader (described below with respect to FIG. 4) is inserted into the channel of the oval spreader until its ends pass deep to the rims of the vertebrae. The spreaders are now intertwined. In this engaged position both spreaders are rotated, in unison, ninety degrees so that the blades of the flat blade spreader are now oriented in a cephalad-caudad direction. The flat blade spreader is moderately dilated by inserting progressively thicker rectangular shaped dilator and the spreading actions exerted on the spreader handles. The oval spreader is then removed.

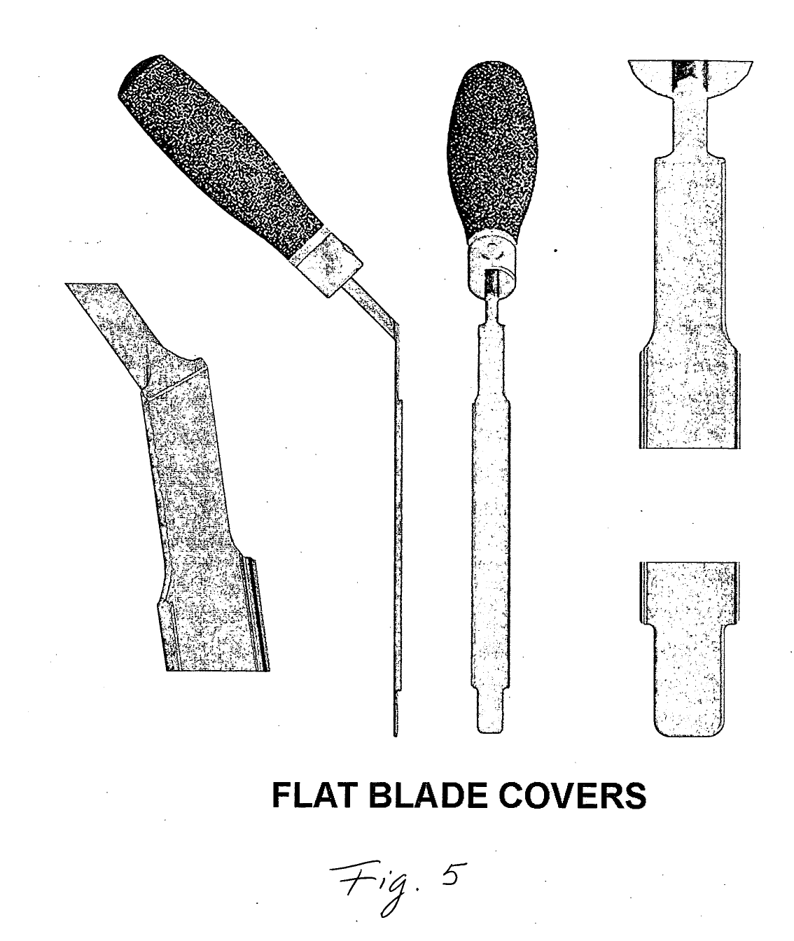

Additional spreading of the flat blade spreader continues by using thicker rectangular dilators. The cephalad and caudad open sides of the flat spreader are closed by preferred flat covers. (See FIG. 5.) The ends of the cover end stay outside of the disc space but engage the outside surfaces of the vertebral body. The multilateral angular walls of the tunnel is assembled by protective flat surfaces. Ultimately, the soft tissue access tunnel from skin into the disc space is rectangular in shape and approximately 8-15 mm. in height and width.

An operating endoscope is now inserted to the end of the beveled cannula. If the pathology is that of intracanal intervertebral disc herniation work spaces are created deep into the annulus working tunnel and the working cavity. Biting forceps are positioned to open the herniation annular collar. Once the collar is opened, it can be removed through the previously established work spaces.

If the operating pathology calls for fusion, the following steps are followed. From the plain x-ray of the spine in two views, the surgeon measures the disc height. The surgeon also estimates the further height distractible from bending films. The preferred tapered obturator of such diameter estimated from the above measures is then employed. A single step dilation of the disc space is carried out using this preferred tapered spiral obturator. The spiral ridge will aid advance and dilate the annular opening by rotating movement.

When the taper end of the obturator fully enters the disc space, the disc space height distraction reaches anatomical maximum. An oval spreader is slid over the tapered obturator and engages the vertebral rim. The oval spreader is opened further passively using solid bore rods until the spreader blades can achieve a 3-4 mm. further opening. At this point, the rotational orientation of the oval spreader blades is such that the convex center of each blade engages the bony rims of the opposing vertebra, and the blade opening is parallel to the disc. Up to this step, the fenestration made in the annulus remains circular in shape. In the methodology for fusion preparation, when the disc distraction has reached its maximum limits and all of the nucleus pulposus and cartilagenous end plate have been removed, the excavated cavity is roughly the shape of a disc(biconvex and round).

The annular opening thus far is circular in its gross dimensions. The shape of the circular annular fenestration can be changed, in the subsequent steps, to an angular opening by the unique methodology of the present invention. In the preferred embodiment, the circular shaped opening is changed to a multilateral angular opening in the shape of a square or rectangle using the flat-blade spreader as discussed above. The angular shaped opening wastes no distracted disc space height dimension and will accept the angular implant components for maximum size and contact surfaces between the graft and the host bed.

With respect to end plate preparation, multiple shallow perforations are made in the subchondral bone of both end-plates to allow for the entry of a blood supply for the fusion process.

The configuration of implant graft to be inserted is so designed as to achieve the largest possible surface area and height that is in contact with the opposing host end-plates surface. The implant material should be tall enough so that the graft/end-plate surfaces are under compression. The ideal vertebral interbody implant shape is that of a disc. Since the access tunnel from the skin into the disc space is very limited in height and width, it is preferred to modularize the whole discoid shape implant into two or more components to facilitate the passage of the material through the relatively smaller access tunnel.

After the insertion of the graft material, osteoconductive and osteoinductive supplementary agents in the form of paste, jelly, granules, or sponge can also be inserted to fill any small crevices or voids that remain in the target intervertebral disc space.

Referring next to FIG. 2, the preferred embodiment of the tapered spiral-end obturator 220 is shown. The tapered spiral-end obturator 220 is used initially to create a tunnel in the patient's soft tissue. This allows the surgeon to gain access to the intervertebral disc space of interest that is to be prepared using one of the preferred surgical methods described. The taper spiral-end obturator 220 is preferably manufactured from stainless steel so as to have sufficient strength, and to permit multiple sterilizations. Alternatively, the obturator may be manufactured from a different type of metal such as titanium. Other materials can also be used. For example, the obturator can be manufactured from a hard plastic material. Manufacturing the obturator out of plastic is particularly advantageous when the obturator is intended to be disposed of after each procedure, rather than being sterilized and re-used.

The tapered spiral-end obturator 220 has a generally elongated cylindrical portion 221. One end 223 of the obturator is tapered to a point as shown in FIG. 2. The end is tapered at a central angle of approximately 30 degrees in the preferred embodiment, although other angles can be used with equal effectiveness. The tapered end includes a raised helical ridge 223. The helical ridge 223 is used to assist the surgeon in advancing the obturator 220 into the patient. The tapered spiral-end obturator 220 can be manufactured in a range of different diameters and lengths. In the preferred embodiment, the tapered spiral-end obturator 220 has a diameter of approximately 6-14 mm and a length of approximately 15-25 cm.

Referring next to FIG. 3, the preferred embodiment of the beveled canulum 200 is shown. The beveled canulum 200 is used to expand and retract the patient's soft tissue so as to allow access to the spinal vertebrae and discs. The beveled canulum 200 is preferably manufactured from stainless steel so as to have sufficient strength, and to permit multiple sterilizations. Alternatively, the canulum is manufactured from another metal such as titanium. Other materials can also be used. For example, the canulum can be manufactured from a hard plastic material. Manufacturing the canulum out of plastic is particularly advantageous when the device is intended to be disposed of after use, rather than being sterilized and re-used. The canulum can be manufactured from a clear plastic material. A clear plastic tube can transmit light into the intervertebral disc region, making it easier for the surgeon to view the area of interest. Similarly, the canulum 200 can include a light source (not illustrated in FIG. 3) coupled to it in order to achieve a similar result.

The canulum has a generally elongated cylindrical portion 201 and a beveled end 202 as shown in FIG. 3. The end is beveled at an angle of approximately 35 degrees in the preferred embodiment, although other angles can be used with equal effectiveness. The canulum 200 is hollow so as to permit passage of the various other surgical instruments used during the preferred procedure, as described above.

The canulum 200 can be manufactured in a range of different diameters and lengths so as to gain access into the disc space. During a typical surgical procedure progressively larger diameter cannula can be inserted over each other until an annular opening of the desired size is achieved. In the preferred embodiment, the cannula range in diameter from 7 mm to 16 mm. the cannula will typically have a length of approximately 15-20 cm.

FIG. 4 illustrates a flat-blade spreader that is used in the present invention. FIG. 5 illustrates a cover for the flat blade created tissue tunnel. Several attachment mechanisms, cover to flat blade, are possible with the present invention. In one embodiment, a channel is fabricated into the outer surface of the paired flat blades, allowing for the attachment of covers for the open sides of the flat blade spreader. Alternative attachment mechanisms include clasps and screw-on devices. These fixed attachment methods permit the spreader blades and covers to move as one unit.

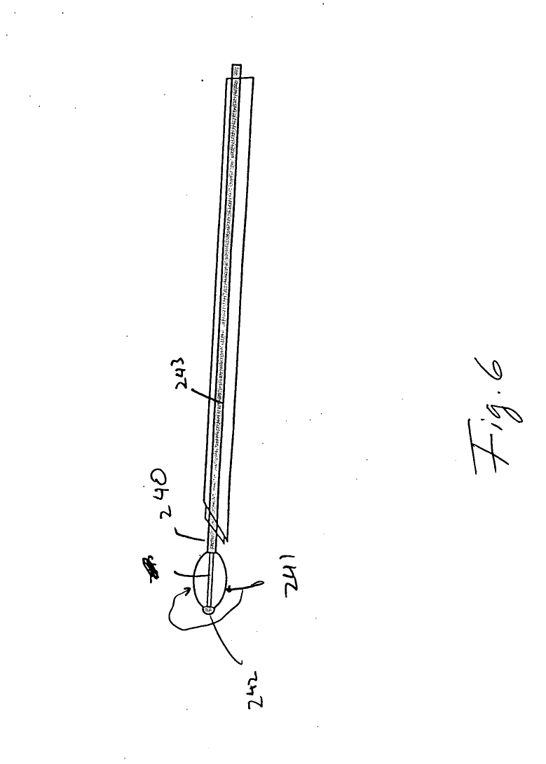

The nucleus pulposus of a disc and its adjacent cartilaginous end-plates require a variety of different tools to achieve complete excision. U.S. patent application Ser. No. 09/997,361 describes several tools that are available for performing this process. Several additional tools are illustrated in FIGS. 6 through 8.

Referring next to FIG. 6, a nucleus debriding tool 240 is illustrated. The debriding tool 240 is shown having passed through a beveled cannulum 200. The debriding tool includes an elongated shaft 243. Shaft 243 is of sufficient length to permit the debriding tool to pass completely through the beveled cannulum 200. Attached at one end of the shaft 243 is a rod 244. A pair of cutting wires 241 are coupled to the shaft 243 at one end and the distal end of the rod 244 at the other end. The cutting wires are of a length slightly greater than the length of rod 244. This results in the cutting wires forming a small loop as shown in FIG. 6. The cutting wires 241 are made from a flexible material such as copper wire, and the exact types of materials will be known to those of skill in the art. This permits the wires to pass through the inside diameter of the beveled cannulum, and then “spring back” into their original shape. Attached to the end of the rod 244 is an insulated tip 242.

The debriding tool operates by passing a small electrical current through the cutting wires 241. The electrical current causes the wires to heat up. The surgeon will then use the wires to cut through any disc material which may need removal. The insulated tip 242 is present to guard against the unintended removal of healthy tissue. The electrical current may be introduced to the cutting wires in a variety of different methods. For example, the wires can pass through the shaft 243 and couple to an electrical source. In an alternative embodiment, the cutting energy may be introduced by a radio-frequency generator. All of the available methods are well-known in the prior art and will be well known to those of skill in the art. The preferred diameter of the debriding tool is 7-14 mm. when the loop formed by the cutting wires 241 is fully expanded.

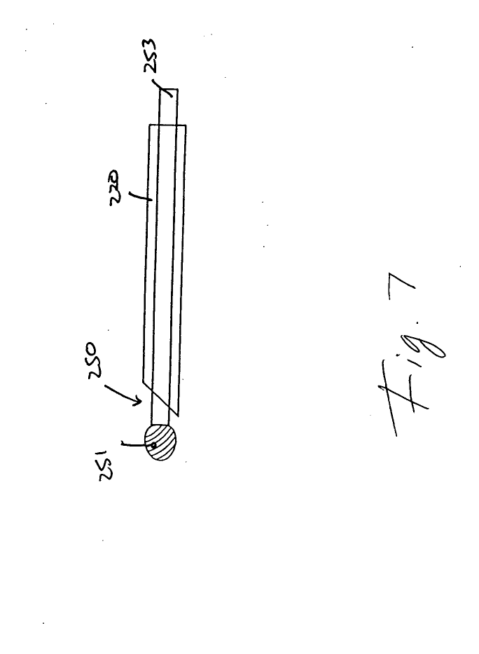

An abrasive tool 250 according to the system of the present invention is illustrated in FIG. 7. The abrasive tool 250 consists of an abrasive head 251 mounted on an elongated shaft 253. The abrasive tool 250 is shown having been passed through a beveled cannulum 200. The abrasive tool 250 includes an elongated shaft 243. Shaft 243 is of sufficient length to permit the abrasive tool to pass completely through the beveled cannulum 200. The abrasive head 251 is used to remove cartilaginous end-plate and can also perforate bony subchondral plates. It can operate in a number of different ways. In one embodiment, the abrasive head 251 acts as a grinder to mechanically remove material. The head is spun either by hand or by being attached to a low speed, high torque power tool. In an alternative embodiment, the abrasive head is heated electronically, and removes tissue by burning and/or melting.

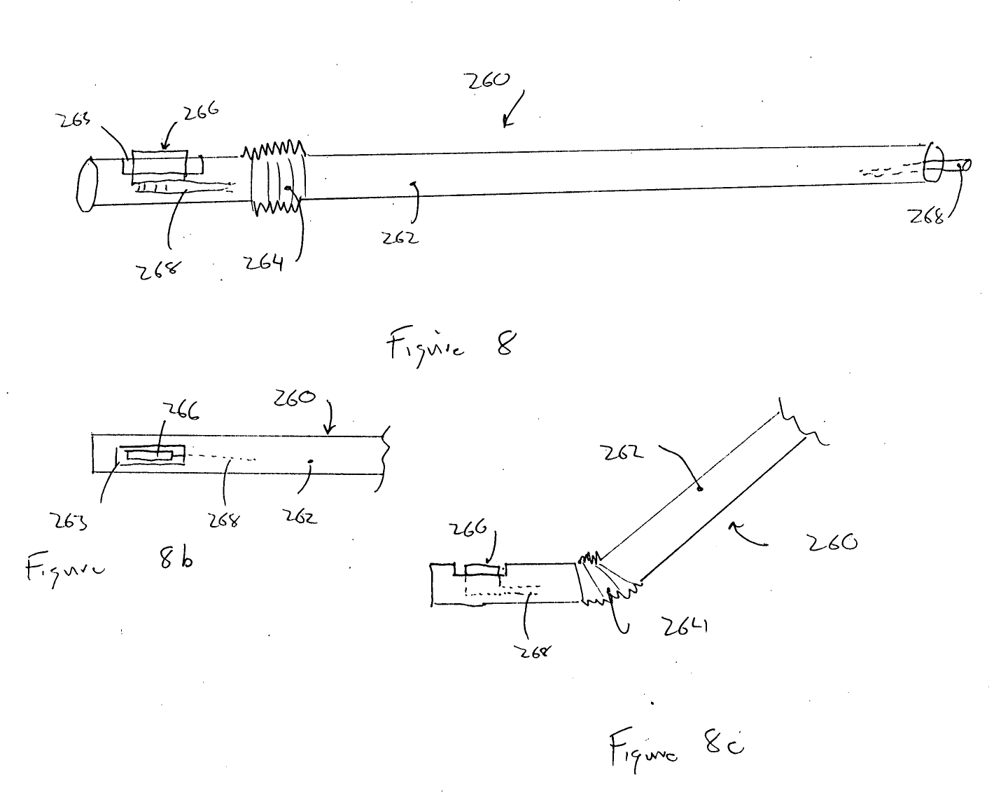

Referring next to FIG. 8, a hollow shaving tool 260 that is used to evacuate the intervertebral disc space is shown. The shaving tool 260 consists of a hollow elongated tube 262. The tube has a diameter small enough to permit it to pass through the soft tissue tunnel created using the spreaders and beveled cannula as described above. The tube also has sufficient length to reach the intervertebral disc space of interest. Disposed on the side of the tube 262 near one end is an opening 263. The opening is best illustrated in FIG. 8b which is a top view of the shaving tool. The shaving tool 260 may also include a flexible portion 264 (similar to that of a bendable drinking straw) to permit the opening 264 to be positioned near the disc space of interest. FIG. 8c shows the shaving tool in its bent configuration.

Located within the shaving tool 260 is a razor knife edge 266. The knife edge 266 is positioned so that it is near the opening 263. The knife edge is coupled to rotational rod 268 that passes through the hollow shaft 262. In alternative embodiments there may be two or more knife edges 266 coupled to the rotational rod 268. Suitable bearings and supports (not shown in FIG. 8) are provided to position the rotational rod 268 within the hollow shaft 260 and support it while it rotates. A suitable flexible joint (not shown) is integrated into the rotational rod 268 to accommodate the bending portion 264 of the hollow shaft 262. The distal end of the rotational rod 268 is coupled to a power source which will cause it to turn as necessary. The precise implementation of the power source will be well known to those of skill in the art, and is not illustrated in FIG. 8. In operation, the surgeon places the opening 263 of the shaver tool 260 near the material which is to be removed from the intervertebral space. Power is applied to the rotational rod 268 which causes the knife edge 266 to turn. The surgeon can use the knife edge to remove the excess tissue, which can then be evacuated through the hollow shaft using a suitable vacuum source if desired.

Accordingly, a system of tools that can be used for minimal access endoscopic spinal surgery have been described. It will be apparent to those skilled in the art that the foregoing description is for illustrative purposes only, and that various changes and modifications can be made to the present invention without departing from the overall spirit and scope of the present invention. The full extent of the present invention is defined and limited only by the following claims.

1. A system for minimal access soft tissue dilating and retracting and nucleus pulposus excision tools for endoscopic spinal surgery, comprising elements to seek the appropriate trajectory, creation of soft tissue tunnel space, and retractors for the tunnels.

2. A tapered spiral-end obturator, comprising:
a generally elongated cylindrical portion 221;
a tapered end coupled to said generally elongated cylindrical portion;
a raised helical ridge disposed on said tapered end.

3. A beveled canulum, comprising a generally elongated cylindrical portion coupled to a beveled end, and being substantially hollow.

4. A debriding tool, comprising:
an elongated shaft;
a rod coupled to said elongated shaft;
a pair of cutting wires, said wires being coupled to said shaft at a first end to said rod at a second end, wherein said wires have a length slightly greater than a length of said rod so as to form a loop;
an insulated tip coupled to an end of said rod;
means for connecting said wires to an electrical source.

5. An abrasive tool comprising:
an elongated shaft;
an abrasive ball coupled to one end of said elongated shaft.

6. A hollow shaving tool, comprising
a hollow elongated tube having an opening disposed on the side of said tube proximate to one end of said tube;
a rotational rod positioned within said elongated tube;
a razor knife edge coupled to said rotational rod and positioned near said opening; and
a power source coupled to said rotational rod for causing said rotational rod to rotate.
